(19) United States
(12) Patent Application Publication
(10) Pub. No.: US 2016/0015657 A1
(43) Pub. Date: Jan. 21, 2016

(54) USE OF EUGENOL TO TREAT BLISTERS

(71) Applicant: Wanda Joy Smith, Williamson, GA (US)

(72) Inventor: Wanda Joy Smith, Williamson, GA (US)

(21) Appl. No.: 14/803,004

(22) Filed: Jul. 17, 2015

Related U.S. Application Data
(60) Provisional application No. 62/025,910, filed on Jul. 17, 2014.

(51) Int. Cl. A61K 31/085; A61K 36/61; A61K 36/185

(52) U.S. Cl. CPC A61K 31/085; A61K 36/185; A61K 36/61

(57) ABSTRACT

Implementations of a method for treating an affected area of an epidermis having a blister or rash caused by shingles, psoriasis, acne, eczema, bug bits, bee stings, and poison oak comprising applying topically to the affected area of an epidermis a composition comprises eugenol and sesame oil.

### CROSS REFERENCE TO RELATED APPLICATION

This application claims the benefit of U.S. patent application Ser. No. 62/025,910, which was filed on Jul. 17, 2014, and is incorporated herein by reference in its entirety.

### TECHNICAL FIELD

This disclosure relates to implementations of a method to use eugenol to treat blisters.

### BACKGROUND

U.S. Pat. No. 8,273,719 disclose that it is known that the eugenol is generally used as an analgesic agent for toothache and others, and further has a blood flow promotion effect or demelanizing effect when applied to a surface topical site. The '719 patent discloses an antiwrinkle agent comprising an eugenyl glycoside.

U.S. Pat. No. 6,313,329 discloses that clove bud oil has long been used as a herbal remedy. The '329 patent discloses that the oil is distilled from the dried-flower buds. The '329 patent discloses that the oil is used in many natural-based toothpastes. The '329 patent discloses that the oil is also a strong insect repellent and useful as a moth repellent. The '329 patent discloses that clove bud oil is a strong antiseptic, anti-spasmodic. The '329 patent discloses that clove bud is also anti viral, anti fungal and healing. The '329 patent discloses that the oil comes from the buds of cloves. The '329 patent discloses that the oil has been used traditionally to remedy skin infections and to reduce digestive upsets. The '329 patent discloses that the oil is also used to kill intestinal parasites and to aid in childbirth. The '329 patent discloses that a tea that is made from cloves is often used to relieve nausea. The '329 patent discloses that the bud oil also has been used for the symptoms above for diarrhea, hernias, bad breath and bronchitis. The '329 patent discloses that the oil can be used to reduce acne, athlete's foot, and pain from burns. The '329 patent discloses that the oil is a very effective insect repellent and will relieve the pain of most toothaches, ulcers and wounds. The '329 patent discloses that the vapors of the oil have beneficial effects on arthritis, rheumatism and most sprains.

### DETAILED DESCRIPTION

A method to use eugenol to treat blisters is provided. In some implementations, the composition may be comprised of 85% or about 85% of eugenol by weight. In some implementations, the composition may be comprised of less than 85% or more than 85% eugenol by weight. In some implementations, the composition may be comprised of 85% or about 85% of eugenol by volume. In some implementations, the composition may be comprised of less than 85% or more than 85% eugenol by volume.

In some implementations, the composition may be comprised of 85% or about 85% of clove oil by weight. In some implementations, the composition may be comprised of less than 85% or more than 85% clove oil by weight. In some implementations, the composition may be comprised of 85% or about 85% of clove oil by volume. In some implementations, the composition may be comprised of less than 85% or more than 85% clove oil by volume.

In some implementations, the composition may be comprised of 15% or about 15% of sesame oil by weight. In some implementations, the composition may be comprised of less than 15% or more than 15% sesame oil by weight. In some implementations, the composition may be comprised of 15% or about 15% of sesame oil by volume. In some implementations, the composition may be comprised of less than 15% or more than 15% sesame oil by volume.

In some implementations, eugenol is a phenylpropene. In some implementations, eugenol is a member of the phenylpropanoids class of chemical compounds. In some implementations, eugenol may be extracted from essential oils. In some implementations, eugenol may be extracted from clove oil, nutmeg, cinnamon, basil, and/or bay leaf.

In some implementations, the composition containing eugenol, and in some implementation sesame oil, may be a paste. In some implementations, the composition containing eugenol, and in some implementation sesame oil, may be a liquid. In some implementations, the composition containing eugenol, and in some implementation sesame oil, may be a gel.

In some implementations, a composition comprising eugenol, and in some implementation sesame oil, may be applied topically to the epidermis of a patient. In some implementations, a composition comprising eugenol, and in some implementation sesame oil, may be applied to a blister which has formed on the epidermis of a patient. In this way, the eugenol, and in some implementation sesame oil, may cause the blister to heal faster than a blister that has not had eugenol applied thereto. In some implementations, the composition comprising eugenol, and in some implementation sesame oil, may be applied topically to the epidermis of a patient to help treat shingles, psoriasis, acne, eczema, irritations from bug bits, bee stings, poison oak, and other skin irritations, rashes, and blisters.

Table 1 shows an example implementation of the composition comprised of eugenol and sesame oil according to the present disclosure. The composition may be comprised of the following ingredients:

TABLE 1

| Components | Quantity |
|---|---|
| Clove Oil | 4.92 ml |
| Sesame Oil | 1.08 ml |

The above composition was used on various skin condition including shingles, acne, eczema, irritations from bug bits, bee stings, poison oak, and other skin irritations, rashes, and blisters. Subjects found the composition eased pain and dried up blisters. The sesame oil helped moisturize the skin and dilute the eugenol.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A composition, comprised of
a first composition comprising an effective amount of eugenol; and
a second composition comprising an effective amount of sesame oil.

2. The composition of claim 1, wherein the first composition is comprised of clove oil.

3. The composition of claim 2, wherein the amount of clove oil is about 85% by volume and the amount of sesame oil is about 15% by volume, based on the volume of the composition.

4. A method for treating shingles comprising applying topically to a shingles blister on an epidermis the composition of claim 3.

5. A method for treating psoriasis comprising applying topically to a psoriasis blister on an epidermis the composition of claim 3.

6. A method for treating blisters comprising applying topically to a blister on an epidermis the composition of claim 3.

7. A method for treating acne comprising applying topically to acne on an epidermis the composition of claim 3.

8. A method for treating eczema comprising applying topically to eczema on an epidermis the composition of claim 3.

9. A method for treating bee stings comprising applying topically to bee sting affected area of an epidermis the composition of claim 3.

10. A method for treating bug bites comprising applying topically to a bug bite affected area of an epidermis the composition of claim 3.

11. A method for treating an affected area of an epidermis having a blister or rash comprising applying topically to the affected area of an epidermis the composition of claim 3.
